(19) United States
(12) Patent Application Publication
(10) Pub. No.: US 2003/0036637 A1
(43) Pub. Date: Feb. 20, 2003

(54) Purification of human serum albumin

(76) Inventor: Scott Fulton, Middleton, WI (US)

Correspondence Address: LOUIS MYERS, Fish & Richardson P.C., 225 Franklin Street, Boston, MA, 02110-2804, US

(21) Appl. No.: 10/172,159

(22) Filed: Jun. 13, 2002

Related U.S. Application Data
(60) Provisional application No. 60/297,884, filed on Jun. 13, 2001.

(51) Int. Cl. C07K 14/765

(52) U.S. Cl. 530/363; 800/7

(57) ABSTRACT

The invention features methods of purifying human serum albumin (hSA) from endogenous serum albumin of the host cell producing the hSA. The methods include providing a sample comprising hSA and serum albumin of the host cell, applying the sample to an affinity column that binds hSA at a higher affinity than the serum albumin of the host cell, eluting bound hSA from the affinity column, and crystallizing the eluted has. The invention also features compositions comprising hSA produced by the methods of the invention.

### RELATED APPLICATIONS

This application claims priority to U.S. Ser. No. 60/297,884, filed on Jun. 13, 2001, the contents of which are incorporated herein by reference.

### BACKGROUND OF THE INVENTION

Serum albumins are one of the most abundant proteins present in blood. They function within the blood as carriers of hydrophobic molecules such as fatty acids and as scavengers that bind to organic molecules, sequestering such molecules until they can be eliminated. Because of their abundance in the blood, serum albumins are a major determinant of the properties of blood and, in particular, blood serum.

During World War II, it was recognized that hSA can be formulated in a physiologically appropriate solution to make a human blood substitute (artificial blood) useful for replacing blood volume lost due to trauma or surgery. See, e .g., Cohn et al. (1946), J. Amer. Chem. Soc. 68:459-75. In fact, such artificial blood is an ideal blood substitute in most cases because the body can readily replace lost blood cells and there is no issue of blood type compatibility. In addition, solutions of hSA have a much longer shelf life than actual blood. Due to the high concentration of hSA in blood, though, large quantities of highly purified hSA are required to produce even a small quantity of artificial blood.

Recombinant production of hSA in transgenic animals is appealing because of the large amount of protein that can be quickly obtained, thereby making it possible to produce significant quantities of artificial blood. Unfortunately, recombinantly produced hSA is not readily useful: it must first be purified away from the serum albumins of the host animal, as well as from other molecules present in the host sample, such as lipids, small molecules, proteins, and viral pathogens. Consequently, at the present time, the process of producing artificial blood-grade hSA starting from samples obtained from transgenic animal sources is both time consuming and costly. At least in part, this is because of the difficulty of separating hSA from the highly similar serum albumins present in animals amenable to use as transgenic hosts. Thus, although recombinant production of hSA in transgenic animals holds the potential of providing large quantities of pure hSA and artificial blood, economic factors have limited its feasibility.

### SUMMARY OF THE INVENTION

The invention is based, in part, on the discovery of a separation method that can distinguish between human serum albumin (hSA) and the serum albumin of a host cell, e.g., a transgenic host cell, e.g., from a transgenic dairy animal. Transgenic production of hSA can result in a product in which hSA and the animal's endogenous serum albumin are both present. It is often necessary, however, to obtain purified hSA that is free of contaminants, especially serum albumins originating from non-human animals. The purification of hSA from a sample obtained from a transgenic animal, e.g., a transgenic dairy animal, can be complicated because there is a high level of homology between hSA and the serum albumins of such animals. For example, hSA is very similar to bovine serum albumin (BSA). Surprisingly, it has been found that transgenically produced hSA can be suitably and efficiently purified from the serum albumin of a host cell using a protocol that includes clarifying a sample containing hSA and an endogenous serum albumin, affinity chromatography with a resin that selectively binds hSA, and crystallization of the hSA following elution from the affinity column.

Accordingly, in one aspect, the invention features a method of purifying human serum albumin (hSA) from a sample that contains hSA and serum albumin of a host cell comprising:

obtaining a sample from a host cell that contains hSA and serum albumin of a host cell;

applying the sample to an affinity column that binds hSA., e.g., binds hSA at a higher affinity than the serum albumin of the host cell;

eluting bound hSA from the affinity column; and

crystallizing the eluted hSA.

In some embodiments, the sample is obtained from a transgenic non-human animal. The animal can be a mammal, e.g., an ungulate (e.g., a cow, goat, or sheep), pig, mouse or rabbit. The sample can be obtained, e.g., from the milk, blood, or tissue (e.g., as a tissue homogenate) of the mammal. In other embodiments, the sample is obtained from a bird, e.g., a chicken, turkey, duck, pheasant, or ostrich. For example, the sample can be obtained from the egg, blood, or tissue (e.g., as a tissue homogenate) of the bird. In preferred embodiments, the animal is a mammal and the sample is milk.

In other embodiments, the sample is medium that has been used to culture cells, e.g., mammalian cells, avian cells, fish cells, or insect cells. In some embodiments, the cultured cells are transgenic cells. For example, the cultured cells can comprise a transgene that comprises a nucleic acid sequence encoding hSA under the control of suitable regulatory elements.

In some embodiments, the sample used in the methods of the invention is milk that has been decreamed, e.g., by a standard decreaming process such as centrifugation.

In related embodiments, the sample used in the methods of the invention is milk (e.g., decreamed milk) that has been treated to remove casein. For example, casein levels in milk can be depleted by reducing the pH of the milk such that a heavy precipitate containing casein forms. In preferred embodiments, the pH of the milk is reduced by adding acid, e.g., a dilute acid, e.g., dilute acetic acid, to the milk. In preferred embodiments, the pH of the milk is reduced to about pH 4.2 to 4.8. In some embodiments, the heavy precipitate of casein is removed from the milk by filtration, e.g., tangential flow microfiltration. In other embodiments, the heavy precipitate of casein is removed from the milk by centrifugation. In yet other embodiments, casein is removed from the sample using tangential flow filtration without acid precipitation of the casein.

In some embodiments, the sample used in the methods of the invention can be decreamed milk from which the casein has been depleted. This sample is also referred to herein as a “clarified milk sample”.

The clarified hSA sample can be subjected to affinity chromatography or can be subjected to one or more additional purification procedures prior to subjecting the sample to affinity chromatography. In some embodiments, the clarified hSA sample is in a salt buffer suitable for loading the affinity column. For example, the salt buffer can include, e.g., 250 mM NaCl at pH 8.5 and a low concentration of a non-ionic detergent.

In some embodiments, the methods of the invention include the use of an affinity column that binds to the hSA protein present in the hSA sample (e.g., the clarified hSA sample), wherein the affinity column comprises a synthetic resin. A suitable synthetic resin for binding to hSA is found in the Prometic Biosciences Blue SA column, which uses a modified version of the dye Reactive Blue 2 as the affinity ligand. In preferred embodiments, the affinity column, e.g., the synthetic resin affinity column, does not substantially bind to many or even most of the non-hSA proteins (e.g., whey proteins) present in the hSA sample. In a particularly preferred embodiment, the affinity column, e.g., the synthetic resin affinity column, has a lower affinity for (e.g., does not substantially bind to) non-human serum albumin proteins, e.g., mammalian serum albumin proteins, e.g., BSA, as compared to its affinity for hSA.

In related embodiments, the methods of the invention include the use of an affinity column that binds to the hSA protein present in the hSA sample (e.g., the clarified hSA sample), wherein the interaction between the affinity column ligand and hSA can be disrupted by a fatty acid molecule. In preferred embodiments, the fatty acid molecule is caprylate.

In some embodiments, the methods of the invention include washing the affinity column after the hSA sample (e.g., the clarified hSA sample) has been applied to the column. In preferred embodiments, the wash buffer is the same as the loading buffer. A suitable wash buffer includes, e.g., 250 mM NaCl at pH 8.5 and a low concentration of a non-ionic detergent.

In some embodiments, the methods of the invention include the use of an elution buffer to elute hSA proteins bound to the affinity column and thereby produce an affinity-purified hSA sample, wherein the elution buffer does not substantially induce the elution of non-serum albumin proteins bound to the affinity column. For the Prometic Biosciences Blue SA column, a suitable elution buffer can comprise a phosphate buffer and a fatty acid molecule that competes with the affinity ligand of the column for binding to hSA. In some embodiments, the elution buffer includes about 20-50 mM phosphate at about pH 6.0. In some embodiments, the elution buffer includes the fatty acid caprylate, e.g., at a concentration of about 20 mM.

In some embodiments, the methods of the invention include reapplying the affinity-purified hSA sample to the affinity column, washing the hSA bound affinity column and eluting the hSA bound to the affinity column to thereby produce a twice affinity-purified hSA sample. In other embodiments, the affinity purified hSA sample can be reapplied to the affinity column more than once, e.g., a thrice affinity-purified hSA sample.

The affinity-purified hSA sample can then be crystallized or can be subjected to one or more additional purification procedures prior to crystallization.

In some embodiments, the methods of the invention include crystallizing the affinity-purified hSA sample (e.g., one-time or twice affinity-purified hSA sample) by adding a crystallizing agent to the sample. Numerous crystallizing agents can be added to a solution of hSA so as to trigger crystallization, including polyethylene glycol (PEG), ammonium sulfate, and/or phosphate. In preferred embodiments, the crystallizing agent is a phosphate solution. In a preferred embodiment, the crystallizing agent is phosphate which is added to the sample to a final concentration of 2.7 to 2.8 M phosphate. In some embodiments, the crystallizing agent further comprises a fatty acid molecule that binds to hSA, e.g., caprylate. In preferred embodiments, the crystallized hSA protein is separated from the crystallization solution (i.e., the mother liquor) by, e.g., filtration, washed in buffer, and redissolved in an appropriate solvent (e.g., water). In other embodiments, the crystallized hSA protein can be dried, e.g., using a solvent or air drying. The dried hSA protein crystal can then be stored, e.g., for extended periods of time, e.g., at room temperature, and optionally, transported. In some embodiments, the dried crystallized hSA can then be redissolved in an appropriate solvent (e.g., water).

In another aspect, the invention features a method of separating hSA from serum albumin of another species. The method includes:

obtaining an hSA sample which further includes serum albumin of another species;

applying the hSA sample to an affinity column that binds has, e.g., binds hSA at a higher affinity than it binds the serum albumin of the other species;

eluting bound hSA from the affinity column; and

crystallizing the eluted hSA.

In some embodiments, the sample is obtained from a non-human animal. The animal can be a mammal, e.g., an ungulate (e.g., a cow, goat, or sheep), pig, or rabbit. The sample can be obtained, e.g., from the milk, blood, or tissue (e.g., as a tissue homogenate) of the mammal. In other embodiments, the sample is obtained from a bird, e.g., a chicken, turkey, duck, pheasant, or ostrich. For example, the sample can be obtained from the egg, blood, or tissue (e.g., as a tissue homogenate) of the bird. In preferred embodiments, the animal is a transgenic animal. In preferred embodiments, the animal is a mammal and the sample is milk, e.g., milk obtained from a transgenic mammal.

In other embodiments, the sample is medium that has been used to culture cells, e.g., mammalian cells, avian cells, fish cells, or insect cells. In some embodiments, the cultured cells are transgenic cells. For example, the cultured cells can comprise a transgene that comprises a nucleic acid sequence encoding hSA under the control of suitable regulatory elements.

In some embodiments, the sample used in the methods of the invention is milk that has been decreamed, e.g., by a standard decreaming process such as centrifugation.

In related embodiments, the sample used in the methods of the invention is milk (e.g., decreamed milk) that has been treated to remove casein. For example, casein levels in milk can be depleted by reducing the pH of the milk such that a heavy precipitate containing casein forms. In preferred embodiments, the pH of the milk is reduced by adding acid, e.g., a dilute acid, e.g., dilute acetic acid, to the milk. In preferred embodiments, the pH of the milk is reduced to about pH 4.2 to 4.8. In some embodiments, the heavy precipitate of casein is removed from the milk by filtration, e.g., tangential flow microfiltration. In other embodiments, the heavy precipitate of casein is removed from the milk by centrifugation. In yet other embodiments, casein is removed from the sample using tangential flow filtration without acid precipitation of the casein.

In some embodiments, the sample used in the methods of the invention can be decreamed milk from which the casein has been depleted. This sample is also referred to herein as a “clarified milk sample”.

In preferred embodiments, the hSA sample is applied to the affinity column, eluted from the affinity column and/or crystallized as described herein.

In another aspect, the invention includes a composition comprising hSA and a serum albumin of a non-human mammal, wherein the serum albumin of the non-human mammal is present at a concentration of less than about 5, 4, 3, 2, or 1 ppm. In preferred embodiments, the ratio of hSA to the serum albumin of the non-human mammal is less than 1:1,000, 1:10,000, 1:100,000.

### DESCRIPTION OF THE DRAWINGS

FIG. 1 depicts a dye ligand chromatographic resin that includes the dye Reactive Blue 2, which is known to bind to serum albumins. The R group can be substituted with a number of different compounds, e.g., —NH—C₆H₄—(meta)SO₃H, —NH—C₆H₄—(ortho)SO₃H, or mixtures thereof. The solid support is agarose.

### DETAILED DESCRIPTION

Decreamed Milk

A milk sample obtained from a transgenic mammal can be decreamed by standard decreaming processes such as centrifugation. The term “decreamed milk” as used herein refers to skim milk. Other known methods including skimming the milk and/or sedimentation to obtain a decreamed sample, see, e.g., H. E. Swaisgood, Developments in Dairy Chemistry, I: Chemistry of Milk Protein, Applied Science Publishers, NY, 1982.

Removal of Casein From the Sample

The methods of the invention can include reducing the level of casein present in the milk sample, e.g., the decreamed milk sample. Preferably, this step can reduce the level of casein in the sample by at least 70%, 80%, 90%, 95% or more as compared to the casein levels in the sample prior to this step.

Casein levels in the sample can be depleted using various methods known in the art. For example, casein levels in a sample can be reduced by acid precipitation. Preferably, the acid is a dilute acid. Examples of acids which can be used to precipitate casein from a sample include acetic acid, sulfuric acid and phosphoric acid. Preferably, the acid is acetic acid. By adding acid to the sample, the pH of the sample is reduced to about 4.0 to 5.5, about 4.1 to 5.2, about 4.2 to 5.0, or about 4.2 to 4.8. The acidified sample can then be subjected to centrifugation or tangential flow filtration to remove the precipitated casein. Acid precipitation of caseins is described in further detail in, e.g., U.S. Pat. No. 4,644,056.

Centrifugation can be performed using various standard bench centrifuges at about 2500 to 500 ×G. In addition, centrifugation can be performed using, e.g., an Alfa Laval or Westphalia continuous flow disk-stack centrifuge. These later centrifuges are especially amendable to process scale centrifugation.

The acidified sample can be subjected to tangential flow filtration by passing the sample through a cross flow filter having a membrane of sufficient pore size to retain at least a portion of the precipitated casein (the “retentate”) while allowing the hSA containing sample to pass through the membrane (the “permeate” or “filtrate”). In tangential flow filtration, the sample to be filtered flows parallel to the membrane filter and the filtrate passes through it. Preferably, the membrane is a hollow fiber cartridge having a mean pore size of about 0.08 to 1.2 μm. Membranes having a mean pore size of about 0.1 to 1.2 μm are commercially available. For example, a Ceramem 0.2 μm ceramic monolith can be used in preferred embodiments. In other embodiments, the hollow fiber cartridge is an A/G Technologies 750 K cutoff hollow fiber. Examples of tangential flow filtration methods can be found, for example, in U.S. Pat. No. 4,644,056.

In other embodiments, the casein levels in the sample can be reduced without acidifying the sample. For example, the sample can be passed through tangential flow microfiltration methods such as those set forth in U.S. Pat. No. 6,268,487.

Regardless of whether or not the sample is acidified prior to tangential flow filtration, at least a portion of the casein should be retained by a membrane, and a significant portion of the hSA will be present in the filtrate. Preferably, at least 30%, 40%, 50%, 60%, 70%, 80%, 90% or more of the hSA in the sample will be present in the filtrate after tangential flow filtration. The filtrate can then be subjected to further steps to purify the hSA.

Affinity Chromatography

Affinity chromatography can be performed in a variety of ways and can include the use of synthetic chemical resins (e.g., dye ligand resins) or protein-coupled resins (e.g., antibody-coupled resins). See, e.g., G. Hermanson et al, Immobilized Affinity Ligand Techniques, New York: Academic Press 1992. Critical parameters to consider when deciding upon what type of resin to use for the purification of a protein of interest, e.g., hSA, include cost of producing the column, scalability of the column, and temporal quality of the column (i.e., the quality of the purification results obtained from the column after repeated use). Protein-coupled resins (e.g., antibody-coupled or peptide binding domain-coupled resins) can be highly specific for a target molecule, often having association constants of around 10⁻⁷ to 10⁻¹⁰ M, but are limited by their cost, scalability, and lifetime. In addition, it can sometimes be difficult to recover the protein of interest once it is bound to a protein-coupled resin without harming the resin in the process. Synthetic chemical resins tend to be cheaper, more readily scaled up, and have a longer lifetime than protein-coupled resins, but they also tend to have less specificity, having association constants of around 10⁻⁵ to 10⁻⁹ M.

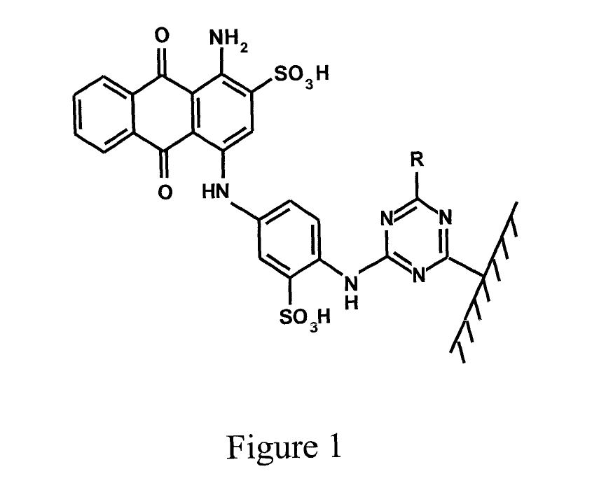

For use in the methods of the invention, an affinity column can comprise either a synthetic chemical resin or a protein-coupled resin. Preferably, the affinity column comprises a synthetic chemical resin. The ligand of the synthetic chemical resin should have an affinity for hSA of at least 10⁻⁵ M, and more preferably at least 10⁻⁶ M, or even 10⁻⁷ M or less. A column suitable for use in the methods of the invention is the Cibacron Blue 3GA column, which is also available from many vendors and has the structure shown in FIG. 1, wherein the R group is a mixture of the structures —NH—C₆H₄—(meta)SO₃and —NH—C₆H₄—(ortho)SO₃H. Another suitable column for use in the methods of the invention is the Prometic Biociences Blue SA column, which is one of a family of dye ligand columns having the resin structure shown in FIG. 1. In one aspect, the dye ligand column is a variant of the Cibacron Blue 3GA column, e.g., a column wherein the R group is not a mixture but is all —NH—C₆H₄—(meta)SO₃H or —NH—C₆H₄—(ortho)SO₃H. Preferably, the synthetic chemical resin has an affinity for hSA that is at least 2, 5, 10, 20, 50, or even 100-fold greater than its affinity for other serum albumins, e.g., non-human mammalian serum albumins, e.g., BSA.

Loading buffers appropriate for loading a sample containing hSA onto a column will depend upon the specific column. In addition, those skilled in the art will recognize that there are many different buffers suitable for loading an hSA sample onto any particular column. A preferred loading buffer for the Prometic Biosciences Blue SA column includes, e.g., 50 to 250 mM salt (e.g., NaCl or KCl), at pH 8-9, and a low concentration of a non-ionic detergent (e.g., 0.01% to 0.1% Polysorbate 20 (i.e., Tween 20)). A clarified hSA sample is preferably diafiltered prior to being loading onto a column in order to exchange the buffer of the clarified hSA sample for an appropriate column loading buffer. A clarified hSA sample can also be filtered so as to increase the concentration of hSA protein in the sample.

Suitable wash buffers for the column are essentially the same as (e.g., identical to) suitable loading buffers. The presence of detergent (e.g., Polysorbate 20) in the wash buffer helps to remove non-human serum albumins (e.g., BSA) from the column (e.g., the Prometic Biosciences Blue SA column) without disrupting the interaction between hSA and the column.

Similarly, elution buffers for eluting hSA from a column to which it is bound will depend upon the exact nature of the column. As those skilled in the art will recognize, there are also many different buffers that are suitable for eluting hSA from a particular column to which it is bound. In the case of the Prometic Biosciences Blue SA column, a suitable elution buffer includes, e.g., 30 to 50 mM phosphate (e.g., a mixture of potassium phosphate and sodium phosphate), at pH 5 to 7, or preferably pH 5.5 to 6.5, and 10-30 mM caprylate. Other fatty acid molecules can be used in place of caprylate, including, e.g., short, medium, or long-chain fatty acids, e.g., stearate, laurate, myristate, and oleate. Preferably, the particular elution buffer used elutes hSA more readily than other non-serum albumin proteins bound to the column (e.g., blood protein, milk proteins, or tissue culture proteins) by a factor of 2, 5, 10, 20, 50, 100, or more.

As discussed above, protein-coupled affinity columns can also be used in the methods of the invention. An exemplary protein-coupled affinity column useful for the purification of hSA from a sample containing other non-human serum albumins (e.g., non-human serum albumins, e.g., BSA) comprises a recombinant albumin binding domain (ABD) protein immobilized on a cross-linked agarose resin. Recombinant albumin binding domain protein has been described in Johansson et al, J. Mol. Biol. 1997, 266:859-865. Preferably, an ABD column has an affinity for hSA that is at least 10, 20, 50, 100, 500, or even 1000-fold greater than its affinity for other serum albumins, e.g., non-human mammalian serum albumins, e.g., BSA.

Equilibration and wash buffers suitable for use with an ABD-coupled column can include, e.g., 10-50 mM acetate at pH 4.5 to 6.5, preferably about pH 5.0 to 6.0, and 50-250 mM salt, preferably about 100-150 mM salt. Possible salts include, e.g., NaCl or KCl. Prior to loading, clarified hSA sample should be adjusted to pH 4.5 to 6.5, preferably pH 5.0 to 6.0 This can be done by adding an acid (e.g., dilute sulfuric or phosphoric acid) or, depending upon the pH of the hSA sample, a base (e.g., dilute NaOH) or by buffer exchange (e.g., diafiltration) into the equilibration and wash buffer. The clarified hSA sample can also be filtered to increase the hSA protein concentration prior to being loaded on the column. hSA can be eluted from an ABD-coupled column using a low pH buffer, e.g., having pH 2.3 to 2.8, preferably about 2.5. Possible elution buffers include 25 to 100 mM glycine or 0.2 to 1.0 M acetic acid. Preferably, the elution buffer used elutes hSA more readily than other non-serum albumin proteins bound to the column (e.g., blood protein, milk proteins, or tissue culture proteins) by a factor of 2, 5, 10, 20, 50, 100, or more.

Preferably, at least 85%, 90%, 91%, 92%, 93%, 94%, 95%, 96%, 97%, 98%, or more of the hSA present in a sample that is applied to the affinity column is recovered in the eluate. Similarly, the concentration of non-hSA protein contaminants present in the hSA sample applied to the affinity column is preferably reduced in the eluate by a factor of 5, 10, 100, 1000, or more. Such contaminants can include non-hSA blood proteins (e.g., clotting proteins, apolipo-proteins, growth factors), milk proteins (e.g., non-human mammalian serum albumins (e.g., BSA), β-lactoglobulins, α-lactoglobulins, and antibodies such as IgGs), egg proteins (e.g., lysozyme), or proteins commonly found in conditioned cell culture medium (e.g., BSA or growth factors).

The affinity chromatography step can optionally be repeated as part of the methods of the invention. In such cases, the eluate from the first column run merely has to be diafiltered to exchange the elution buffer for the appropriate column loading buffer. Prior to being diafiltered, the eluate can optionally be filtered to reduce the amount of fatty acid present in the sample and to concentrate the hSA. More than one affinity column can be used in the methods of the invention when the affinity chromatography step is repeated. For example, a synthetic chemical resin column (e.g., the Cibacron Blue C3A column or Prometic Biosciences Blue SA column) can be used in conjunction with a protein-coupled column (e.g., an ABD column). The order in which the columns are used is not critical, although it is preferable to use the synthetic chemical resin column first so as to maximize the lifetime of the protein coupled column.

In some embodiments, the methods of the invention include performing the affinity chromatography continuously, e.g., on a simulated moving bed system.

Crystallization

“Crystallized hSA”, as used herein, refers to a solid state of hSA which can be distinguished from its amorphous solid state. Crystals display characteristics such as a lattice structure and characteristic shapes and optical properties such as refractive index. The determination of hSA as a crystal can be determined by any means including: optical microscopy, electron microscopy, x-ray powder diffraction, solid-state nuclear magnetic resonance (NMR) or polarizing microscopy. Microscopy can be used to determine the crystal length, diameter, width, size and shape, as well as whether the crystal exists as a single particle or is polycrystalline.

Crystals of hSA can be formed by adding salts, PEG and/or organic solvents to a solution containing hSA (e.g., an affinity purified sample of hSA). Inorganic salts which can be used to crystallize hSA include ammonium sulfate, sodium chloride, potassium chloride, sodium phosphate (e.g., dibasic- and/or monobasic sodium phosphate), potassium phosphate (e.g., potassium phosphate monobasic and/or potassium metaphosphate), or mixtures thereof. Preferably, the inorganic salt used to crystallize hSA is sodium phosphate and/or potassium phosphate. A fatty acid molecule (e.g., caprylate or another medium or long-chain fatty acid molecule) can be added to the hSA sample along with the inorganic salt to aid in the crystallization. For example, a solution containing 4M phosphate, pH 6.2 (70:30 v/v mixture of 4M NaH2PO4 and 4M K2HPO4) and 1 to 3 mM caprylate can be gradually added to a sample of hSA at a temperature of 5-15° C. At a final concentration of about 2.7 to 2.8 M phosphate crystallization of hSA occurs.

Preferably, at least 60%, 65%, 70%, 75%, 80%, 85%, 90%, 95%, 98%, or more of the hSA present in a sample that is crystallized is recovered, e.g., in the redissolved sample. In addition, the concentration of non-hSA protein contaminants present in the hSA sample that is crystallized is preferably reduced in the redissolved sample by a factor of 1, 2, 3, 4, 5, 10, 20, or more. Such contaminants can include non-hSA blood proteins (e.g., clotting proteins, apolipo-proteins, growth factors), milk proteins (e.g., non-human mammalian serum albumins (e.g., BSA), b-lactoglobulins, a-lactoglobulins, and antibodies such as IgGs), egg proteins (e.g., lysozyme), or proteins commonly found in conditioned cell culture medium (e.g., BSA and growth factors).

Crystals of hSA can be separated from the mother liquor, e.g., using a funnel (e.g., a Buchner funnel or equivalent device), and washed, e.g., in 2.8 M phosphate buffer, pH 6.2. Isolated hSA crystals can be dried and stored. Alternatively, isolated hSA crystals can be redissolved in a suitable solvent, e.g., water or a dilute salt solution compatible with parenteral administration (e.g., NaCl).

Storage

At various points in the purification, purified hSA can be filtered (e.g., sterile filtered) and stored in an aseptic container. Such storage can be long-term (e.g., days or months) and amenable to transport. For example, following clarification (i.e., lipid removal and other steps, such as the decreaming of milk and the removal of casein), affinity column purification, crystallization, or treatment of the hSA with activated carbon.

Parenteral Formulations

The hSA sample prepared as described herein can be incorporated into pharmaceutical compositions. Such compositions typically include hSA and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. A preferred route of administration for hSA is parenteral administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of hSA calculated to produce the desired therapeutic effect in association with the required pharmaceutical carrier.

### EXAMPLES

### Example 1

### Purification of hSA from the Milk of a Transgenic Cow

Starting with decreamed (skim) transgenic milk containing the recombinant hSA product, the casein was precipitated by acidifying the product stream to pH 4.2-4.8 with acetic acid (10-15%). The precipitated casein was removed by tangential flow microfiltration using standard TFF systems with cartridges which include Ceramem 0.2 μm ceramic monolith or A/G Technologies 750 K MW cutoff hollow fiber.

The product was purified by affinity chromatography using the Prometic Biosciences Blue SA column. Loading/wash buffer conditions included pH 8-9, 50-250 mM ionic strength NaCl, and a low concentration of Polysorbate 20 (Tween 20). The product was eluted with a 20-50 mM phosphate buffer containing 10-30 mM caprylate at pH 5.5-6.5.

The product was crystallized in a batch tank by controlled addition of 4 M phosphate pH 6.2 (70:30 v/v mixture of 4 M NaH2PO4 and 4 M K2HPO4) and 1-3 mM caprylate to a final phosphate concentration of 2.7-2.8 M at a temperature of 5-15° C. The crystals were separated from the mother liquor by filtration using a Buchner funnel, washed in 2.8 M phosphate buffer, prepared as described above and redissolved in water. The results are shown below in Table 1.

TABLE 1
Purification of hSA

| | | Typical | | | | |
|---|---|---|---|---|---|---|
| | | Step | ppm | ppm | ppm | ppm |
| Process Step | | hSA Yield | bSA | BLG | ALA | IgG |
| 1. | Decreamed milk | 100% | 1 × 10⁵ | 5 × 10⁶ | 1 × 10⁶ | 7 × 10⁵ |
| 2. | Acid casein | 95% | ND | ND | ND | ND |
| | precipitation | | | | | |
| 3. | 1^{st} Dye-ligand | 95% | 350 | 1000 | 550 | 175 |
| | chromatography | | | | | |
| 4. | 2^{nd} Dye-ligand | 95% | 15 | 50 | 25 | 50 |
| | chromatography | | | | | |
| 5. | Crystallization | 80% | 5 | 5 | <0.4 | <0.4 |

Purity measurements by individual protein ELISA assays.
ppm—parts per million (μg contaminant per g hSA)
bSA—bovine serum albumin
BLG—β-lactoglobulin
ALA—α-lactalbumin
IgG—gamma globulin G

### Example 2

### Purification of hSA using Two Different Affinity Columns

Starting with decreamed (skim) transgenic milk containing the recombinant hSA product, the sample was clarified by acid precipitating casein as described in Example 1. Subsequently, dye-ligand affinity chromatography was performed as described above.

Finally, ABD protein-ligand chromatography was performed on the dye-ligand affinity purified eluate. The ABD column equilibration and wash buffer used included 10-50 mM acetate, pH 5.0-6.0, and 100-150 mM NaCl. Prior to loading the ABD column, the pH of the hSA sample was adjusted to pH 5.0-6.0 by the addition of dilute acid. The hSA was eluted with a 25-100 mM glycine buffer, pH 2.5. The results of the purification are shown in Table 2.

TABLE 2

| | | Typical | | |
|---|---|---|---|---|
| | | Step | | |
| Process Step | | hSA Yield | ppm bSA | ppm IgG |
| 6. | Clarified feedstream | 99% | 2 × 10⁴ | 1 × 10⁴ |
| 7. | Dye-ligand | 95% | 300 | 30 |
| | chromatography | | | |
| 8. | ABD protein-ligand | 95% | 3 | 3 |
| | chromatography | | | |

The contents of all publications and patents cited herein are incorporated by reference.

What is claimed is:

1. A method of purifying human serum albumin (hSA) from a sample that contains hSA and serum albumin of a host cell comprising:
obtaining a sample from a host cell that contains hSA and serum albumin of a host cell;
applying the sample to an affinity column that binds hSA at a higher affinity than the serum albumin of the host cell;
eluting bound hSA from the affinity column; and
crystallizing the eluted hSA.

2. The method of claim 1, wherein the sample is obtained from a transgenic non-human animal.

3. The method of claim 2, wherein the animal is selected from the group consisting of a cow, a sheep, a goat, a pig, a mouse and a rabbit.

4. The method of claim 2, wherein the sample is obtained from the milk, blood, or tissue of the mammal.

5. The method of claim 1, wherein the sample is medium that has been used to culture cells.

6. The method of claim 2, wherein the sample is obtained from the milk of a transgenic mammal which produces hSA in its mammary epithelial cells.

7. The method of claim 6, wherein the method further comprises decreaming the milk sample.

8. The method of claim 6, wherein the method further comprises treating the milk sample to remove casein.

9. The method of claim 8, wherein the casein is removed by acid precipitation, centrifugation or tangential flow filtration.

10. The method of claim 6, wherein the sample is a clarified milk sample.

11. The method of claim 10, wherein the clarified milk sample is in a salt buffer.

12. The method of claim 11, wherein the salt buffer comprises 250 mM NaCl at pH 8.5 and a low concentration of a non-ionic detergent.

13. The method of claim 11, wherein the affinity column comprises a synthetic ligand resin.

14. The method of claim 13, wherein the synthetic ligand resin uses a dye Reactive Blue 2 or a modified dye Reactive Blue 2 as an affinity ligand.

15. The method of claim 1, wherein the affinity column does not substantially bind to the serum albumin of the host cell as compared to its affinity to bind hSA.

16. The method of claim 1, further comprising washing the affinity column after the sample has been applied to the column.

17. The method of claim 16, wherein the wash buffer comprises 250 mM NaCl at pH 8.5 and a low concentration of a non-ionic detergent.

18. The method of claim 1, wherein the hSA is eluted from the affinity column using an elution buffer does not substantially induce the elution of non-serum albumin proteins bound to the affinity column.

19. The method of claim 18, wherein the elution buffer comprises a phosphate buffer and a fatty acid molecule that competes with the affinity ligand of the column for binding to hSA.

20. The method of claim 19, wherein the elution buffer comprises about 20-50 mM phosphate at about pH 6.0.

21. The method of claim 19, wherein the fatty acid molecule is caprylate.

22. The method of claim 1, further comprising applying the affinity-purified hSA sample to the affinity column or a second affinity column, washing the hSA bound affinity column and eluting the hSA bound to the affinity column to thereby produce a twice affinity-purified hSA sample.

23. The method of claim 1, wherein the bound hSA is crystallized by adding a crystallizing agent to the sample.

24. The method of claim 23, wherein the crystallizing agent is selected from the group consisting of polyethylene glycol (PEG), ammonium sulfate, phosphate, or combinations thereof.

25. The method of claim 23, wherein the crystallizing agent is phosphate and is added to a final concentration of 2.7 to 2.8 M.

26. The method of claim 23, wherein the crystallizing agent further comprises a fatty acid molecule that binds to hSA.

27. The method of claim 27, wherein the fatty acid molecule is caprylate.

28. The method of claim 1, wherein the crystallized hSA is separated from the crystallization solution.

29. A composition comprising hSA made by the method of claim 1.
